(19) United States
(12) Patent Application Publication
(10) Pub. No.: US 2010/0083425 A1
(43) Pub. Date: Apr. 8, 2010

(54) Eyewear

(76) Inventor: Austen Miller, Whitchurch, Hampshire (GB)

Correspondence Address: BRACEWELL & GIULIANI LLP, P.O. BOX 61389, HOUSTON, TX, 77208-1389, US

(21) Appl. No.: 12/593,049

(22) PCT Filed: Mar. 28, 2008
(86) PCT No.: PCT/GB08/50223

§ 371 Date: Dec. 9, 2009

(30) Foreign Application Priority Data
Mar. 28, 2007 (GB) 0705967.8

(51) Int. Cl. A61F 9/02

(52) U.S. Cl. 2/426

(57) ABSTRACT

Protective eyewear, for example swimming goggles, is known to possess a nose bridge. Known adjustment mechanisms for the nose bridge either require a mechanism that cannot be adjusted in situ, i.e. when the goggles are in use, and so is inconvenient, or that does not reliably maintain the eyepieces over the eyes of a wearer, for example when the wearer dives into water. The present invention therefore provides eyewear (100) comprising a first eyepiece (102) and a second eyepiece (104). The first eyepiece (102) is coupled to the second eyepiece (104) by a cord (114). At least one of the first and second eyepieces (102, 104) is translatable, when in use, relative to the cord (114) upon overcoming a translation resisting force.

The present invention relates to eyewear of the type, for example, comprising a first eyepiece and a second eyepiece that are secured over eyes by a strap extending around the back of a head.

In the field of protective eyewear for swimmers, a number of designs of swimming goggles are known. In this respect, each design typically has a drawback that can be inconvenient for a given swimmer.

For example, one known design of swimming goggles comprises a first eyepiece and a second eyepiece, each having a peripheral guide disposed around an upper arc and a lower arc thereof; the peripheral guides have one or more respective spaced peripheral restriction. Each eyepiece is disposed within a respective loop of an elastomeric strap so that an upper arc and a lower arc of each loop passes through the peripheral guides of each eyepiece. The loops meet at a frontal “X” formation and each loop is integrally formed with an elastomeric band that extends, when in use, behind a head. Each loop comprises longitudinally spaced compressible nodules that are sized so as not to pass easily through the peripheral restrictions. The impediment provided by the peripheral restrictions of the eyepieces to the nodules result in each eyepiece being substantially maintained in situ relative to the elastomeric strap. When placed over the eyes, with the strap extending around and behind the head, the eyepieces can be adjustably aligned with respective eyes by pulling each eyepiece in a lateral translational manner with respect the face of a wearer in order urge one or more nodule of each loop against neighbouring peripheral restrictions, thereby causing the nodules to compress and pass through the peripheral restrictions. However, due to the elasomeric nature of the strap, the strap has a tendency to stretch, particularly when the wearer of the goggles dives into water, resulting in the displacement of one or both of the eyepieces relative to the eyes and hence depriving the eyes of protection. Clearly, this drawback is inconvenient and can be extremely disadvantageous in competitive sports.

EP-B1-1 106 214 relates to swimming goggles comprising: two eyepieces and a first joining unit and a second joining unit on two opposite ends of each eyepiece. At least one “round string”, pulled in sequence through the first and the second joining units of the two eyepieces, maintains an appropriate span between the two eyepieces. A headband is also joined in series to the two joining units of outside edges of the eyepieces. The span of the round string between the eyepieces can be adjusted so as to adjust the span of the nose bridge. However, adjustment of the round string is inconvenient, requiring unhooking the round string from at least one clasp mechanism; this adjustment cannot be achieved easily whilst the goggles are in situ over the eyes and so removal of the goggles is required in order to adjust the separation of the eyepieces.

According to the present invention, there is provided eyewear comprising a first eyepiece coupled to a second eyepiece by a cord, at least one of the first and second eyepieces being translatable, when in use, relative to the cord upon overcoming a translation resisting force.

The translation resisting force may result from interaction of a length of the cord with the at least one of the first and second eyepieces. The length of the cord may be of substantially uniform transverse cross-section.

The cord may follow a path defined by the at least one of the first and second eyepieces, the path being substantially free of obstacles. The cord may be a single length of cord. The cord may be substantially inelastic.

The translation resisting force may be a latchless translation resisting force.

The eyewear may further comprise a spacing between the first and second eyepieces, the spacing being maintained, when in use, by the translation resisting force.

The spacing may be adjustable by overcoming the translation resisting force.

The translation resisting force may be overcome, when in use, by longitudinal translational movement of the at least one of the first and second eyepieces relative to the cord. The translational movement may be induced by a translational force sufficiently large to overcome the translation resisting force. The translational force may be provided, when in use, by finger manipulation of the at least one of the first and second eyepieces.

The translation resisting force may be overcome by drawing the at least one of the first and second eyepieces along the cord.

The translation resisting force may be overcome by drawing the first and second eyepieces together or drawing the first and second eyepieces away from each other.

The cord may be formed as a single loop. The loop may extend from a first side of the first eyepiece and return to the first side of the eyepiece so as to form a first side loop with the first eyepiece.

The loop may extend from a second side of the second eyepiece and returns to the second side of the eyepiece so as to form a second side loop with the second eyepiece.

The cord may be anchored to the first eyepiece. The cord may be anchored to the second eyepiece.

The cord may be anchored by providing the cord with an enlargement. The enlargement may be a knot, or a bead formed on the cord.

The at least one of the first and second eyepieces may channel the cord. The at least one of the first and second eyepieces may channel the cord so as to form the single loop.

The cord may be channelled so as to assume a meandering path. The first eyepiece and/or second eyepiece may cause the cord to assume a bending path relative to the first eyepiece and/or the second eyepiece. The first eyepiece and/or the second eyepiece may comprise a respective conduit therethrough.

The translation resisting force may be produced by interaction of a length of the cord with the respective conduit. The interaction may be between an outer surface of the cord and an inner surface of the respective conduit when the cord is under tension. The respective conduit may comprise a kink or a dogleg. The cord may extend through the respective conduit.

The enlargement may be greater than the conduit. The first and/or second eyepiece may comprise a recess in order to receive the enlargement. The recess may be provided in a path of the respective conduit.

The translation resisting force may be achieved, when in use, by tensioning the cord within the conduit.

The cord and the first and second eyepieces may be arranged so that, when in use, a translational movement of the first eyepiece in a first direction permits reciprocal translational movement by the second eyepiece in a second, opposite, direction. The translational movement of the first eyepiece may result in a variation in an available length of the cord to extend between the first and second eyepieces, thereby facilitating the reciprocal translational movement of the second eyepiece.

The eyewear may further comprise a tensioner. The tensioner may be coupled to the cord. In this respect, the tensioner may be coupled to the first side loop and/or the second side loop. The tensioner may be a strap, for example formed from an elastomeric material.

The Eyewear may be swimming goggles.

It is thus possible to provide eyewear, for example swimming goggles, which can be adjusted with ease whilst placed over the head. Additionally, the eyepieces do not displace when a wearer dives into water. Furthermore, fewer adjustable parts are required to achieve adjustment of separation of the eyepieces. In this respect, adjustment of the nose bridge is achieved in a latchless of claspless manner.

At least one embodiment of the invention will now be described, by way of example only, with reference to the accompanying drawings, in which:

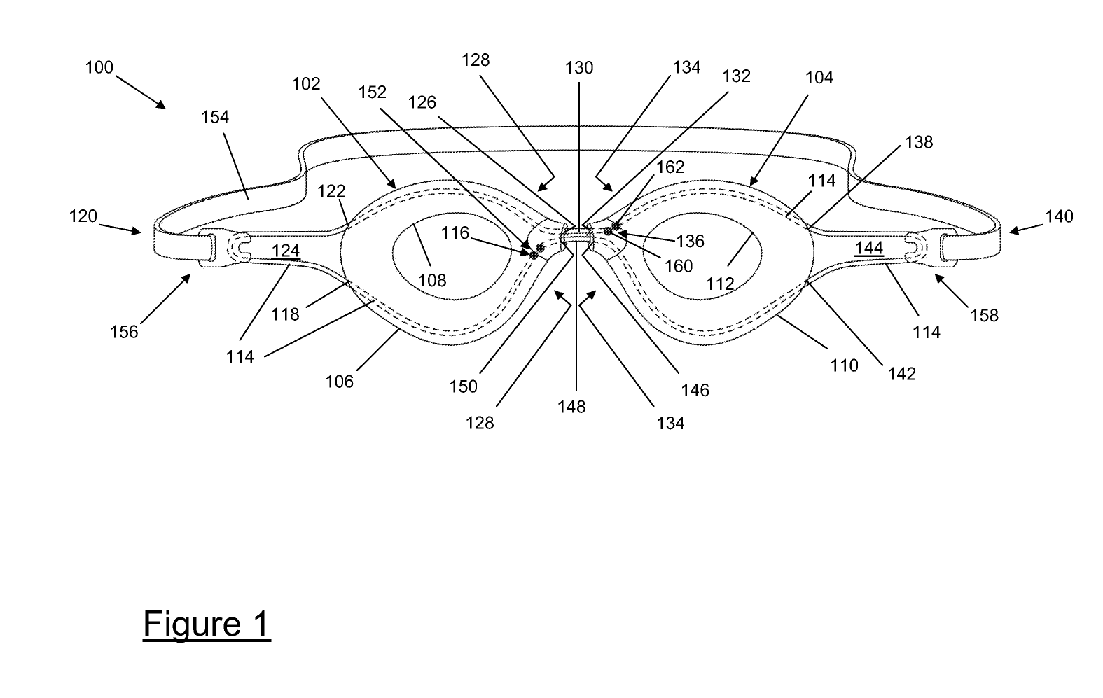

FIG. 1 is a schematic diagram of swimming goggles constituting an embodiment of the invention; and

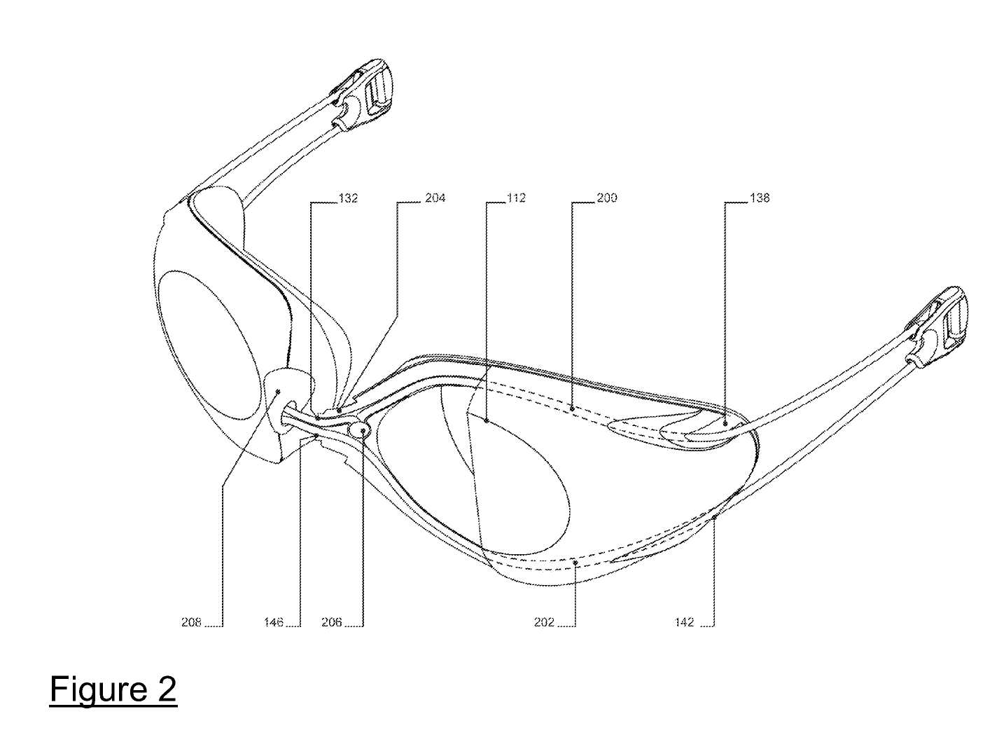

FIG. 2 is a schematic diagram of an eyepiece of the swimming goggles of FIG. 1 in greater detail.

Throughout the following description identical reference numerals will be used to identify like parts.

Referring to FIG. 1, a pair of swimming goggles 100 comprises a first eyepiece 102 and a second eyepiece 104. The first eyepiece 102 comprises a first backing piece 106 and a first lens cover 108, and the second eyepiece 104 comprises a second backing piece 110 and a second lens cover 112. The first eyepiece 102 is coupled to the second eyepiece 104 by a cord 114. The cord is, in this example, flexible and of substantially uniform transverse cross-section, for example a length of string. The cord 114 is substantially inelastic and does not stretch sufficiently to result in displacement of the first or second eyepiece 102, 104, when in use, from a position over one or both eyes of a wearer of the swimming goggles. The cord 114 is, in this example, of a single length. In this example, the cord 114 has been threaded through each of the first and second eyepieces 102, 104 as a single loop in the following manner.

Starting from an initial point 116 in the first eyepiece 102, the cord 114 passes around and under the first lens cover 108 and exits the first eyepiece 102 via a first outer port 118 at a first outer side 120 of the first eyepiece 102. The cord 114 then returns and re-enters the first eyepiece 102 via a second outer port 122 also at the first outer side 120 so as to form a first side loop 124 with the first eyepiece 102. The cord 114 then passes around and over the first lens cover 108 before exiting the first eyepiece 102 at a first inner port 126 at a first inner side 128 of the first eyepiece 102.

The cord 114, having exited the first eyepiece 102, extends to the second eyepiece 104 via a first bridging length 130 of the cord 114 and enters the second eyepiece 104 at a second inner port 132 at a second inner side 134 of the second eyepiece 104. The cord 114 then passes via a first anchoring point 136 and continues around and over the second lens cover 112 before exiting the second eyepiece 104 via a third outer port 138 at a second outer side 140 of the second eyepiece 104.

The cord 114 then returns and re-enters the second eyepiece 104 via a fourth outer port 142 also at the second outer side 140 so as to form a second side loop 144 with the second eyepiece 104. Thereafter, the cord 114 passes around and below the second lens cover 112 and exits the second eyepiece 104 via a third inner port 146 at the second inner side 134. The cord 114, having exited the second eyepiece 104, extends back to the first eyepiece 102 via a second bridging length 148 of the cord 114 and enters the first eyepiece 102 at a fourth inner port 150 at the first inner side 128 of the first eyepiece 102. The cord 114 then passes back to the initial point 116, which is also a second anchoring point 152. Hence, it can be seen that the first and second eyepieces 102, 104 channel the cord 114 along a predetermined path relative to the first and/or second eyepieces 102, 104, the path being substantially obstacle-free.

An elastomeric strap 154 is coupled at a first end 156 thereof to the first side loop 124 and at a second end 158 thereof to the second side loop 144 by respective fasteners. The fasteners permit the length of the elastomeric strap 154 to be adjustable at each of the first and second ends 156, 158 thereof, for example by a respective buckle-like arrangement or fastener.

Turning to FIG. 2, the second eyepiece 104 will now be described in greater detail. For the sake of conciseness and clarity of description, the first eyepiece 102 will not be described in the same detail herein, as the skilled person will appreciate that the first eyepiece 102 is simply a like configuration of the second eyepiece 104.

In this example, the second backing piece 110 and the second lens cover 112 are formed by any suitable extrusion process so as to form, when sandwiched together, the second eyepiece 104 with a first upper conduit 200 and a first lower conduit 202 disposed therein. The first upper conduit 200 joins the third outer port 138 to the second inner port 132, the first lower conduit 202 joining the fourth outer port 142 to the third inner port 146.

The second backing piece 110 and the second lens cover 112, when sandwiched together, define a first nozzle-like portion 204 in which a recess 206 formed in the second backing piece 110 can be concealed. When assembled, a cover sleeve 208 is affixed over the nozzle-like portion 204 in order to hide the recess 206, the recess 206 being located at the first anchoring point 136 in the path of the first upper conduit 200.

In this respect, a first end 160 of the cord 114 is knotted or provided with a thermoplastic bead (not shown) formed thereon in order to prevent passage of the first end 160 of the cord 114 through the first upper conduit 200 and escape, or detach from, the second eyepiece 104. The enlargement at the first end 160 of the cord 114 is therefore blocked when the cord 114 is under tension, due to its size being greater than the diameter of the first upper conduit 200, from passing through the first upper conduit 200 and hence remains in the recess 206.

A second end 162 of the cord 114 is also knotted or provided with another thermoplastic bead (not shown) formed thereon in order to prevent passage of the second end 162 of the cord 114 through the first upper conduit 200 and escape, or detach from, the second eyepiece 104. Consequently, the first and second ends of the cord 114 are anchored in the second eyepiece 104 at the first anchoring point 136.

At the second anchoring point 152, another recess (not shown) is provided in a corresponding second nozzle-like portion (not shown) of the first eyepiece 102 in the same manner as in relation to the second eyepiece 104, though the another recess is disposed in the path of a second lower conduit (not shown) in the first eyepiece 102. Approximately halfway along the length of the cord 114, the cord 114 is either knotted or provided with yet another thermoplastic bead formed thereon so as to anchor the centre of the cord 114 at the second anchoring point 152. This is achieved as the size of the knot or the thermosplastic bead is greater than the diameter of the second lower conduit of the first eyepiece 102 and so remains in the recess of the first eyepiece 102 when the cord 114 is under tension, thereby preventing the centre of the cord 114 from travelling along the second lower conduit.

From the above description of the first and second anchoring points 136, 152, it can be seen that, in this example, the first and second anchoring points 136, 152 are disposed in substantially opposite relation to each other.

In use, the length of the strap 154 is adjusted using the fasteners at the first and second side ends 156, 158 of the strap 154 in order to accommodate the size of the head (not shown) of the wearer. Typically, the first and second eyepieces 102, 104 are then placed approximately over the forehead of the wearer and the strap 154 is drawn over the back of the head of the wearer. The first and second eyepieces 102, 104 are then pulled away from the head slightly and placed over, and in registry with, the eyes of the wearer.

By virtue of the passage of the strap 154 around the back of the head and the elastic nature of the strap 154, a tension is applied to the cord 114 at the first and second side loops 124, 144 thereof, thereby resulting in the cord 114 being pulled around the sides of the head. Consequently, the parts of the cord 114 passing through the conduits in the first and second eyepieces 102, 104 abut and frictionally engage the inner walls of the conduits, resulting in a translation resisting force being generated. This translation resisting force is assisted by the conduits defining a meandering, or bending, path. The meandering or bending path can include a kink or a dogleg. The translation resisting force resists lateral translational movement of the eyepieces 102, 104 across the face of the head and hence substantially maintains relative spacing between the first and second eyepieces 102, 104. However, application, typically using finger grip, of a sufficiently large translational force by the wearer results in the translation resisting force being overcome and the eyepiece being permitted to translate laterally. In an alternative embodiment, the translation resisting force can be provided by a clip, clasp, latch or a like mechanism.

Due to the anchoring of the cord 114 within the first and second eyepieces 102, 104, the translational movement of the first and second eyepieces 102, 104 relative to the cord 114 is constrained. In this respect, the cord 114 and the first and second eyepieces 102, 104 constitute a closed system. A first point of coupling of the first end 156 of the strap 154 with the first side loop 124 of the cord 114 constitutes a first point of circumvolution and a second point of coupling of the second end 158 of the strap with the second side loop 144 constitutes a second point of circumvolution.

Consequently, adjustment initiated by lateral translational movement of the first eyepiece 102 away from the second eyepiece 104 results in circumvolution of some of the cord 114 about the first point of circumvolution at the first outer side 120 due to the anchoring of the cord 114 at the first anchoring point 136 in the second eyepiece 104 and the tension applied to the cord 114 by the strap 154. The translational movement of the first eyepiece 102 away from the second eyepiece 104 results in the size of the first side loop 124 decreasing and the length the first bridging length 130 increasing. The increase in the length of the first bridging length 130 permits the second eyepiece 104 to move away from the first eyepiece 102. Due to the anchoring of the cord 114 at the second anchoring point 152 and the tension applied to the cord 114 by the strap 154, the translational movement of the first eyepiece 102 and hence the second anchoring point 152 away from the second eyepiece 104 results in circumvolution of some of the cord 114 about the second point of circumvolution at the second outer side 140. The first anchoring point 136 and hence the second eyepiece 104 are therefore drawn away from the first eyepiece 102, the size of the second side loop 144 decreasing to accommodate a consequential increase in the length of the second bridging length 148. Consequently, translational divergence between the first eyepiece 102 and the second eyepiece 104 takes place, i.e. the first and second eyepieces 102, 104 are drawn apart.

Likewise, adjustment initiated by lateral translational movement of the second eyepiece 104 away from the first eyepiece 102 results in circumvolution of some of the cord 114 about the second point of circumvolution at the second outer side 140 due to the anchoring of the cord 114 at the second anchoring point 152 in the first eyepiece 102 and the tension applied to the cord 114 by the strap 154. The translational movement of the second eyepiece 104 away from the first eyepiece 102 results in the size of the second side loop 144 decreasing and the length the second bridging length 148 increasing. The increase in the length of the second bridging length 148 permits the first eyepiece 102 to move away from the second eyepiece 104. Due to the anchoring of the cord 114 at the first anchoring point 136 and the tension applied to the cord 114 by the strap 154, the translational movement of the second eyepiece 104 and hence the first anchoring point 136 away from the first eyepiece 102 results in circumvolution of some of the cord 114 about the first point of circumvolution at the first outer side 120. The second anchoring point 152 and hence the first eyepiece 102 are therefore drawn away from the second eyepiece 104, the size of the first side loop 124 decreasing to accommodate a consequential increase in the length of the first bridging length 130. Consequently, translational divergence between the first eyepiece 102 and the second eyepiece 104 takes place, i.e. the first and second eyepieces 102, 104 are drawn apart.

Conversely, adjustment initiated by lateral translational movement of the first eyepiece 102 towards the second eyepiece 104 results in circumvolution of some of the cord 114 about the first point of circumvolution at the first outer side 120 due to the anchoring of the cord 114 at the second anchoring point 152 in the first eyepiece 102 and the tension applied to the cord 114 by the strap 154. The translational movement of the first eyepiece 102 towards the second eyepiece 104 results in the size of the first side loop 124 increasing and the length the first bridging length 130 decreasing. The decrease in the length of the first bridging length 130 permits the second eyepiece 104 to move towards the first eyepiece 102. Due to the anchoring of the cord 114 at the second anchoring point 152 and the tension applied to the cord 114 by the strap 154, the translational movement of the first eyepiece 102 and hence the second anchoring point 152 results in circumvolution of some of the cord 114 about the second point of circumvolution at the second outer side 140. The first anchoring point 136 and hence the second eyepiece 104 are therefore drawn towards the first eyepiece 102, the size of the second side loop 144 increasing to accommodate a consequential decrease in the length of the second bridging length 148. Consequently, translational convergence between the first eyepiece 102 and the second eyepiece 104 takes place, i.e. the first and second eyepieces 102, 104 are urged together.

Likewise, adjustment initiated by lateral translational movement of the second eyepiece 104 towards the first eyepiece 102 results in circumvolution of some of the cord 114 about the second point of circumvolution at the second outer side 140 due to the anchoring of the cord 114 at the first anchoring point 136 in the second eyepiece 104 and the tension applied to the cord 114 by the strap 154. The translational movement of the second eyepiece 104 towards the first eyepiece 102 results in the size of the second side loop 144 increasing and the length the second bridging length 148 decreasing. The decrease in the length of the second bridging length 148 permits the first eyepiece 102 to move towards the second eyepiece 104. Due to the anchoring of the cord 114 at the first anchoring point 136 and the tension applied to the cord 114 by the strap 154, the translational movement of the second eyepiece 104 and hence the first anchoring point 136 results in circumvolution of some of the cord 114 about the first point of circumvolution at the first outer side 120. The second anchoring point 152 and hence the first eyepiece 102 are therefore drawn towards the second eyepiece 104, the size of the first side loop 124 increasing to accommodate a consequential decrease in the length of the first bridging length 130. Consequently, translational convergence between the first eyepiece 102 and the second eyepiece 104 takes place, i.e. the first and second eyepieces 102, 104 are urged together.

The first and second bridging lengths 130, 148 serve as a nose bridge for the goggles 100, the nose bridge thus being adjustable in the manner described above. Adjustment of the relative positions of the first and second eyepieces 102, 104 is latchless.

Although the above embodiment has been described in the context of swimming goggles, the skilled person will appreciate that the above described configuration can be applied to other suitable eyewear, for example, other protective eyewear.

What is claimed is:

1. Eyewear comprising a first eyepiece coupled to a second eyepiece by a cord, at least one of the first and second eyepieces being translatable, when in use, relative to the cord upon overcoming a translation resisting force.

2. Eyewear as claimed in claim 1, wherein the translation resisting force results from interaction of a length of the cord with the at least one of the first and second eyepieces.

3. Eyewear as claimed in claim 2, wherein the length of the cord is of substantially uniform transverse cross-section.

4. Eyewear as claimed in any one of the preceding claims claim 1, wherein the cord follows a path defined by the at least one of the first and second eyepieces, the path being substantially free of obstacles.

5. (canceled)

6. (canceled)

7. (canceled)

8. (canceled)

9. (canceled)

10. (canceled)

11. (canceled)

12. (canceled)

13. Eyewear as claimed in claim 1, wherein the cord is formed as a single loop.

14. Eyewear as claimed in claim 13, wherein the loop extends from a first side of the first eyepiece and returns to the first side of the eyepiece so as to form a first side loop with the first eyepiece.

15. Eyewear as claimed in claim 13, wherein the loop extends from a second side of the second eyepiece and returns to the second side of the eyepiece so as to form a second side loop with the second eyepiece.

16. Eyewear as claimed in claim 1, where the cord is anchored to the first eyepiece.

17. Eyewear as claimed in claim 1, wherein the cord is anchored to the second eyepiece.

18. (canceled)

19. (canceled)

20. Eyewear as claimed in claim 1, wherein the at least one of the first and second eyepieces channel the cord.

21. (canceled)

22. (canceled)

23. (canceled)

24. Eyewear as claimed in claim 1, wherein the first eyepiece and/or the second eyepiece comprise a respective conduit therethrough.

25. Eyewear as claimed in claim 24, wherein the translation resisting force is produced by interaction of a length of the cord with the respective conduit.

26. Eyewear as claimed in claim 25, wherein the interaction is between an outer surface of the cord and an inner surface of the respective conduit when the cord is under tension.

27. (canceled)

28. Eyewear as claimed in claim 24, wherein the cord extends through the respective conduit.

29. (canceled)

30. (canceled)

31. (canceled)

32. Eyewear as claimed in claim 24, wherein the translation resisting force is achieved, when in use, by tensioning the cord within the conduit.

33. Eyewear as claimed in claim 1, wherein the cord and the first and second eyepieces are arranged so that, when in use, a translational movement of the first eyepiece in a first direction permits reciprocal translational movement by the second eyepiece in a second, opposite, direction.

34. Eyewear as claimed in claim 33, wherein the translational movement of the first eyepiece results in a variation in an available length of the cord to extend between the first and second eyepieces, thereby facilitating the reciprocal translational movement of the second eyepiece.

35. (canceled)

36. (canceled)

37. (canceled)

38. (canceled)

39. (canceled)

40. (canceled)

41. Eyewear as claimed in claim 1, wherein the cord is a single length of cord.

42. (canceled)

43. (canceled)

44. (canceled)

45. Eyewear as claimed in claim 13, wherein at least one of the first and second eyepieces channel the cord.

46. Eyewear as claimed in claim 45, where the at least one of the first and second eyepieces channel the cord so as to form the single loop.
